(19) United States
(12) Patent Application Publication
(10) Pub. No.: US 2010/0324282 A1
(43) Pub. Date: Dec. 23, 2010

(54) METHOD FOR SEPARATING HETEROPOLYACID

(75) Inventors: Masayoshi MURAMATSU, Miyoshi-shi (JP); Satoshi Yoneda, Toyota-shi (JP); Tsuruyo Shimazaki, Toyota-shi (JP); Shusei Obata, Nagoya-shi (JP)

Correspondence Address: SUGHRUE MION, PLLC, 2100 PENNSYLVANIA AVENUE, N.W., SUITE 800, WASHINGTON, DC, 20037, US

(73) Assignee: TOYOTA JIDOSHA KABUSHIKI KAISHA, Toyota-shi (JP)

(21) Appl. No.: 12/816,730

(22) Filed: Jun. 16, 2010

(30) Foreign Application Priority Data
Jun. 17, 2009 (JP) 2009-144355

(51) Int. Cl. C07H 1/06

(52) U.S. Cl. 536/127

(57) ABSTRACT

This invention provides a method for separating a heteropolyacid from a monosaccharide in the presence of water. The method comprises separating a heteropolyacid from a mixture containing a monosaccharide, the heteropolyacid and water using an organic solvent selected from the group consisting of linear C₂₋₄ alkyl ethyl ether and linear or branched C₆₋₁₂ alcohol.

### TECHNICAL FIELD

The present invention relates to a method for separating a heteropolyacid from a monosaccharide in the presence of water. More particularly, the present invention relates to a method for separating a heteropolyacid from a reaction mixture obtained by cellulose hydrolysis.

### BACKGROUND ART

Fossil fuels have been heretofore used as automobile fuels; however, fossil fuels generate CO₂ upon combustion, which has become a worldwide problem as an adverse influence on the global warming. Under such circumstances, plant-derived bioethanol is in use these days as a carbon neutral fuel. However, currently available bioethanol is synthesized from food such as sugar or starch, which would disadvantageously induce food shortages in developing countries.

In addition, methods for producing bioethanol from a large quantity of unused biomass resources (e.g., cellulose), in which fuel and food crops would not compete for the same resources, have been studied. For example, a method of hydrolyzing cellulose with sulfuric acid to generate sugar and fermenting the resulting sugar to produce ethanol is known. Since the solubility of sulfuric acid in water is equivalent to that of the sugar generated upon cellulose degradation, it is difficult to separate sulfuric acid from such sugar.

According to JP Patent Publication (kokai) Nos. 2008-271787 A and 2009-60828 A, cellulose hydrolysis is carried out with the use of a heteropolyacid instead of sulfuric acid. After hydrolysis, water is removed from the reaction mixture, and a heteropolyacid is then separated from glucose.

### SUMMARY OF THE INVENTION

### Object of the Invention

According to the past methodology, it was difficult to separate an acid from a monosaccharide in the presence of water. Therefore, the present invention is intended to provide a method for separating a heteropolyacid from a monosaccharide in the presence of water.

### Means for Attaining the Object

The present inventors have conducted concentrated studies in order to attain the above object. As a result, they discovered that such object could be attained by treating an aqueous solution containing a heteropolyacid and a monosaccharide with a given organic solvent.

Specifically, the present invention is summarized as follows.

(1) A method for separating a heteropolyacid from a mixture containing a monosaccharide, the heteropolyacid and water using an organic solvent selected from the group consisting of linear C₂₋₄ alkyl ethyl ether and linear or branched C₆₋₁₂ alcohol.

(2) The method according to (1), wherein the organic solvent is selected from the group consisting of n-butyl ethyl ether, diethyl ether, 2-ethyl-1-hexanol, 1-octanol, 2-octanol and nonanol.

(3) The method according to (1) or (2), wherein the monosaccharide is glucose.

(4) The method according to any of (1) to (3), wherein the mixture is obtained by allowing cellulose to react with a heteropolyacid.

(5) The method according to any of (1) to (4), wherein the heteropolyacid is a phosphotungstic acid.

### EFFECTS OF THE INVENTION

The present invention enables separation of a heteropolyacid from a monosaccharide in the presence of water. Accordingly, such separation can be carried out in a simple manner without the need for the step of removing water prior to the step of separation. Also, the separated heteropolyacid can be reused, and the present invention can thus contribute to cost reduction.

### BRIEF DESCRIPTION OF THE DRAWING

FIG. 1 shows the results of the saccharification reaction of cellulose using a heteropolyacid with the elapse of time.

### EMBODIMENTS FOR CARRYING OUT THE INVENTION

### 1. Monosaccharide

The term “monosaccharide” refers to a saccharide that cannot be further hydrolyzed. The term “monosaccharide” used herein refers to all known monosaccharides. Examples thereof include erythrose, threose, ribose, lyxose, xylose, arabinose, allose, talose, gulose, glucose, altrose, mannose, galactose, idose, erythrulose, xylulose, ribulose, psicose, fructose, sorbose, and tagatose. In the present invention, preferably, the term “monosaccharide” refers to glucose.

In the present invention, a monosaccharide to be separated may be a single type of monosaccharide or combinations of two or more types of monosaccharides. Preferably, the term “monosaccharide” refers to glucose generated upon hydrolysis of cellulose. A very small amount of other monosaccharides may be generated depending on a type of cellulose starting material to be used, and such monosaccharides are within the scope of the present invention.

### 2. Heteropolyacid

The term “heteropolyacid” refers to a polyacid having a polynuclear structure in which two or more types of oxo-acids are condensed. The term “heteropolyacid” used herein refers to all known heteropolyacids, and examples thereof include phosphotungstic acid, silicotungstic acid, phosphomolybdic acid, sodium molybdophosphate, phosphotungstomolybdic acid, and phosphovanadomolybdic acid. In the present invention, the term “heteropolyacid” preferably refers to phosphotungstic acid. According to the present invention, a single type of heteropolyacid or combinations of two or more types of heteropolyacids can be separated from a monosaccharide.

A heteropolyacid contains crystal water. Since a heteropolyacid can be separated from a monosaccharide in the presence of water according to the present invention, such crystal water would not become an issue of concern in the present invention.

A heteropolyacid can be separated from an aqueous solution containing a monosaccharide with the use of an organic solvent for separation described below. The separated heteropolyacid can be reused. For example, such heteropolyacid can be reused as a catalyst for cellulose hydrolysis.

### 3. Organic Solvent for Separation

According to the present invention, a heteropolyacid can be separated from a monosaccharide in the presence of water with the use of a given organic solvent. Specifically, an organic solvent that dissolves a heteropolyacid but does not dissolve a monosaccharide can be used. For example, linear C₂₋₄ alkyl ethyl ether and linear or branched C₆₋₁₂ alcohol can be used. Preferably, n-butyl ethyl ether, diethyl ether, 2-ethyl-1-hexanol, 1-octanol, 2-octanol and nonanol can be used. Such organic solvents can be used alone or in combinations of two or more. Organic solvents can be selected or combined in accordance with monosaccharide and heteropolyacid types. Other known organic solvents can also be added within the scope of the present invention.

### 4. Mixture

The term “mixture” used herein refers to a mixture containing a monosaccharide, a heteropolyacid and water. Water contained in the mixture is, for example, water as a solvent, crystal water of a heteropolyacid, and water contained in the plant resource as a starting material of a monosaccharide.

According to an embodiment of the present invention, the term “mixture” refers to a reaction mixture obtained by the reaction of cellulose and a heteropolyacid. In this reaction, crystal water of a heteropolyacid is used for hydrolyzing cellulose, and the mixture contains a heteropolyacid, glucose as a hydrolysis product of cellulose, and crystal water of a heteropolyacid. Moisture contained in cellulose may be used for hydrolysis. In the case of such mixture, the generated glucose is dissolved in water contained in the mixture.

According to another embodiment of the present invention, the term “mixture” refers to a reaction mixture resulting from the reaction of cellulose and a heteropolyacid using water as a solvent.

Since a heteropolyacid is soluble in water, the heteropolyacid and a monosaccharide are dissolved together in water when the mixture contains water.

An example of the mixture of the present invention is a mixture resulting from the reaction of a heteropolyacid and a resource that serves as a starting material for a monosaccharide. Examples of such resources that can be used include, but are not limited to, waste wood, rice straw, weed, used paper, sugarcane, maize, and bagasse.

### 5. Separation

The mixture may be subjected to extraction with the use of an organic solvent for separation in order to selectively separate a heteropolyacid. A heteropolyacid is preferably extracted at room temperature.

The amount of the organic solvent for separation used in the process of separation is not limited. It is preferable that an organic solvent for separation is used in an amount that is 2 to 4 times, and particularly 3 or 4 times, greater than the minimal amount of the solvent (g) that is able to dissolve the heteropolyacid contained in the mixture at room temperature (hereafter such amount is referred to as “the minimal amount of solvent for dissolution”).

By extracting the residue of the mixture, which had been subjected to extraction once, with the use of an organic solvent for separation again, the percentage of heteropolyacid extraction can be enhanced.

This description includes part or all of the contents as disclosed in the description of Japanese Patent Application No. 2009-144355, which is a priority document of the present application.

### EXAMPLES

Hereafter, the present invention is described in greater detail with reference to the following examples, although the present invention is not limited thereto.

### Example 1

### Test of Cellulose Saccharification by Heteropolyacid Using Conical Flask

Phosphotungstic acid (30 g) was melted and cellulose powder (0.5 g) was added. The resultant was subjected to a heat reaction with stirring. Water was added to the sample 0, 5, 30, and 45 minutes after the initiation of the reaction, and the mixture was subjected to centrifugation and filtration. The supernatant was subjected to sugar analysis via liquid chromatography (HPLC).

### Assay Conditions:

HPLC system: HP Series 1100 (Hewlett-Packard)
Guard column: SHIM-PACK SPR-PB(G), Shimadzu Co. Ltd. Commodity code: P/N 228-35841-95
Column: SHIM-PACK SPR-PB, Shimadzu Co. Ltd. Commodity code: P/N 228-35840-95
Amount of sample injected: 2 μl
Temperature for analysis: 80° C.
Detector (RID): Agilent Technologies Series 1200, Agilent Technologies
Temperature for detection: 40° C.
Carrier solution: water
Flow rate at the time of analysis: 0.6 ml/min
Duration: 23 minutes

### Results

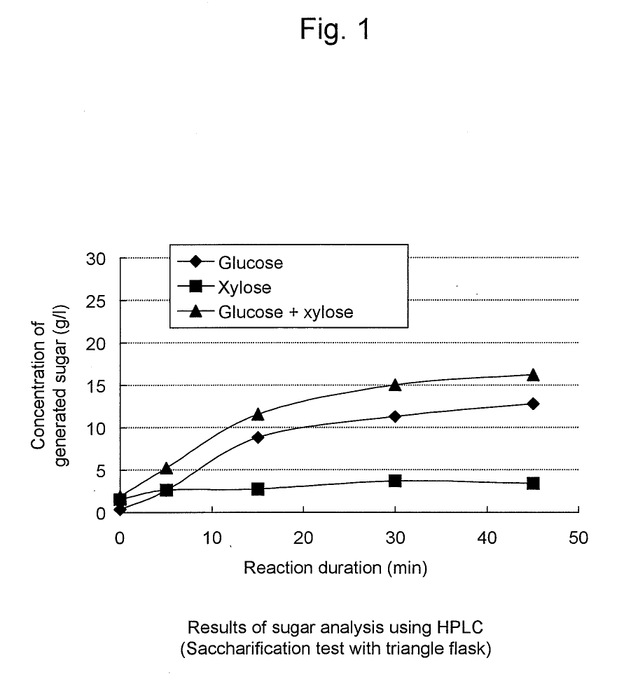

As shown in FIG. 1, glucose and xylose were detected. The amounts of glucose and xylose generated 45 minutes after the initiation of the reaction were about 13 g/l and 3 g/l, respectively.

### Example 2

### Assay of Solubility and Selection of Organic Solvent for Separation

### (i) Assay of Solubility of Phosphotungstic Acid

Phosphotungstic acid was added to an organic solvent at room temperature. Phosphotungstic acid was continuously added until it was not able to dissolve therein, and solubility was determined based on the entire amount of phosphotungstic acid added. The assayed solubility is shown in Table 1.

### (ii) Assay of Solubility of Glucose

Glucose was added to an organic solvent at room temperature. Glucose was continuously added until it was not able to dissolve therein, and solubility was determined based on the entire amount of glucose added. The assayed solubility is shown in Table 1.

TABLE 1
Results of organic solvent selection

| | | Solubility of | | |
|---|---|---|---|---|
| | | phosphotungstic | Solubility of | |
| | Solvents | acid (wt %) | glucose (wt %) | Notes: |
| 1 | Dipentyl ether | x | — | |
| 2 | Diisopentyl ether | x | — | |
| | (isoamyl ether) | | | |
| 3 | Anisole | x | — | Cancelled because |
| | | | | of discoloration |
| | | | | (yellowing) |
| 4 | Phenetole | x | — | |
| 5 | Dibutyl ether | 81.8 | <0.03 | |
| | (n-butyl ether) | | | |
| 6 | Diisopropyl ether | x | — | |
| 7 | n-Butyl ethyl ether | 300 | <0.03 | |
| 8 | Vinyl isobutyl ether | x | | Cancelled because of fever |
| | | | | onset and discoloration |
| | | | | (browning) |
| 9 | Dibenzyl ether | x | — | |
| 10 | Diethyl ether | >229.3 | <0.025 | |
| 11 | n-Hexane | x | — | |
| 12 | Octane | x | — | |
| 13 | Amylbenzene | x | — | Cancelled because |
| | (1-phenylpentane) | | | of yellowing |
| 14 | Propylbenzene | x | — | |
| 15 | Xylene (o-xylene) | x | — | |
| 16 | p-Chlorotoluene | x | — | |
| 17 | Toluene | x | — | |
| 18 | 2-Ethyl-1-hexanol | 158.3 | <0.042 | |
| 19 | 2-Octanol | 200 | <0.043 | |
| 20 | Nonanol | 154 | <0.047 | |
| 21 | Methylene chloride | x | — | |
| | (dichloromethane) | | | |
| 22 | 1-Octanol | 200 | <0.045 | |

“x”: phosphotungstic acid was insoluble; “—”: Not assayed

### Results:

Three types of ethers, i.e., dibutyl ether, n-butyl ethyl ether, and diethyl ether, and four types of alcohols, i.e., 2-ethyl-1-hexanol, 1-octanol, 2-octanol, and nonanol, were found to be promising for separation of phosphotungstic acid from glucose.

### Example 3

### Assay of Distribution Ratio of Phosphotungstic Acid to Organic Solvent and Water

An organic solvent was added to powdery phosphotungstic acid in amounts 2 to 4 times greater than the minimal amount of the solvent necessary to dissolve phosphotungstic acid (see Example 2). The resultant was stirred and allowed to stand, followed by phase separation. Samples were obtained from each phase in an amount of 1 ml and the samples were dried using a centrifuge. The dried specimens were analyzed by the inductively coupled plasma (ICP) mass spectrometer. Water was dissolved in each phase in order to identify an aqueous phase. The results are shown in Table 2.

TABLE 2
Results of assay of distribution ratio of phosphotungstic
acid to organic solvent and water (with the use
of powdery phosphotungstic acid)

| | Phase order | Distribution ratio of | |
|---|---|---|---|
| | (from top to | phosphotungstic acid | Aqueous |
| | bottom) | (based on tungsten) (%) | phase |
| Dibutyl ether | First phase | 0.8 | x |
| | Second phase | 67.6 | x |
| | Third phase | 31.6 | ∘ |
| n-Butyl | First phase | 0.1 | x |
| ethyl ether | Second phase | 2.9 | ∘ |
| | Third phase | 97.0 | x |
| Diethyl ether | First phase | 0.01 | x |
| | Second phase | 99.4 | x |
| | Third phase | 0.6 | ∘ |
| 2-Ethyl-1-hexanol | First phase | 97.3 | x |
| | Second phase | 2.7 | ∘ |
| 2-Octanol | First phase | 97.9 | x |
| | Second phase | 2.1 | ∘ |
| Nonanol | First phase | 96.3 | x |
| | Second phase | 3.7 | ∘ |
| 1-Octanol | First phase | 97.7 | x |
| | Second phase | 2.3 | ∘ |

### Results:

Phosphotungstic acids were detected in the third phase of n-butyl ethyl ether and in the second phase of diethyl ether in amounts of 97% and 99.4%, respectively. Phosphotungstic acids were detected in the first phases of 2-ethyl-1-hexanol, 2-octanol, nonanol, and 1-octanol in amounts of 97.3%, 97.9%, 96.3%, and 97.7%, respectively. The above phases in which most phosphotungstic acids were detected were organic solvent phases.

### Example 4

### Assay of Distribution Ratio of Phosphotungstic Acid to Organic Solvent and Water

An organic solvent was added to a saturated aqueous solution of phosphotungstic acid in amounts 2 to 4 times greater than the minimal amount of the solvent necessary to dissolve the phosphotungstic acid (see Example 2). The resultant was stirred and allowed to stand, followed by phase separation. Samples were obtained from each phase in an amount of 1 ml and the samples were dried using a centrifuge. The dried specimens were analyzed by the ICP mass spectrometer. Water was dissolved in each phase in order to identify an aqueous phase. The results are shown in Table 3.

TABLE 3
Results of assay of distribution ratio of phosphotungstic
acid to organic solvent and water (with the use of saturated
aqueous solution of phosphotungstic acid)

| | Phase order | Distribution ratio of | |
|---|---|---|---|
| | (from top to | phosphotungstic acid | Aqueous |
| | bottom) | (based on tungsten) (%) | phase |
| Dibutyl ether | First phase | 1.1 | x |
| | Second phase | 16.5 | x |
| | Third phase | 82.4 | ∘ |
| n-Butyl | First phase | 0.1 | x |
| ethyl ether | Second phase | 5.1 | ∘ |
| | Third phase | 94.8 | x |
| Diethyl ether | First phase | 0.01 | x |
| | Second phase | 1.5 | ∘ |
| | Third phase | 98.5 | x |
| 2-Ethyl-1-hexanol | First phase | 1.0 | ∘ |
| | Second phase | 98.1 | x |
| | Third phase | 0.9 | ∘ |
| 2-Octanol | First phase | 1.0 | ∘ |
| | Second phase | 97.7 | x |
| | Third phase | 1.3 | ∘ |
| Nonanol | First phase | 1.3 | ∘ |
| | Second phase | 97.3 | x |
| | Third phase | 1.5 | ∘ |
| 1-Octanol | First phase | 1.3 | ∘ |
| | Second phase | 97.3 | x |
| | Third phase | 1.4 | ∘ |

### Results:

Phosphotungstic acids were detected in the third phase of n-butyl ethyl ether and in the third phase of diethyl ether in amounts of 94.8% and 98.5%, respectively. Phosphotungstic acids were detected in the second phases of 2-ethyl-1-hexanol, 2-octanol, nonanol, and 1-octanol in amounts of 98.1%, 97.7%, 97.3%, and 97.3%, respectively. The above phases in which greatest quantities of phosphotungstic acids were detected were organic solvent phases.

### Example 5

### Assay of Distribution Ratio of Glucose

n-Butyl ethyl ether (26.67 ml) was added to a saturated aqueous solution containing 30 g of phosphotungstic acid. The resultant was stirred and allowed to stand, followed by phase separation. Glucose was continuously added until glucose was not able to dissolve in each phase. Solubility of glucose in n-butyl ethyl ether was determined based on the amount of glucose dissolved. The results are shown in Table 4.

TABLE 4
Results of assay of distribution ratio of glucose (with the
use of saturated aqueous solution of phosphotungstic acid)

| | Phase order | Distribution | Distribution ratio of | |
|---|---|---|---|---|
| | (from top to | ratio of | phosphotungstic acid | Aqueous |
| | bottom) | glucose (%) | (based on tungsten) (%) | phase |
| n-Butyl | First phase | 0.1 | 0.1 | x |
| ethyl ether | Second phase | 97.7 | 5.1 | ∘ |
| | Third phase | 2.2 | 94.8 | x |

### Results:

97.7% of glucose was dissolved in the second phase (i.e., the aqueous phase) and 94.8% of phosphotungstic acid was dissolved in the third phase (i.e., the organic phase).

### Example 6

### Assay of Distribution Ratio of Phosphotungstic Acid and Glucose (Heat Experiment)

Phosphotungstic acid triacontahydrate (30 g) was mixed with glucose (5 g), the resultant was heated, and various types of the selected organic solvents were added in amounts 3 times greater than the minimal amount of the solvent necessary to dissolve phosphotungstic acid (by volume). The resultant was stirred and allowed to stand, followed by phase separation. The samples were dried using a centrifuge, the phosphotungstic acid content in the dried specimens was analyzed by the ICP mass spectrometer, and glucose content was assayed via liquid chromatography. The results are shown in Table 5 and in Table 6.

TABLE 5
Results of heating reaction test (with the use
of phosphotungstic acid triacontahydrate)

| | Phase order | Distribution ratio of | Distribution |
|---|---|---|---|
| | (from top to | phosphotungstic acid | ratio of |
| | bottom) | (based on tungsten) (%) | glucose (%) |
| n-Butyl | First phase | 0.1 | — |
| ethyl ether | Second phase | 10.2 | 100 |
| | (aqueous phase) | | |
| | Third phase | 89.7 | 0 |
| Diethyl | First phase | 0.03 | 0 |
| ether | Second phase | 1.7 | 100 |
| | (aqueous phase) | | |
| | Third phase | 98.3 | 0 |

TABLE 6
Results of heating reaction test (with the use of
phosphotungstic acid triacontahydrate and alcohol)

| | Phase order | Distribution ratio of | Distribution |
|---|---|---|---|
| | (from top to | phosphotungstic acid | ratio of |
| | bottom) | (based on tungsten) (%) | glucose (%) |
| 2-Ethyl-1- | First phase | 95.2 | 42.9 |
| hexanol | Second phase | 4.8 | 57.2 |
| | (aqueous phase) | | |
| 2-Octanol | First phase | 98.3 | — |
| | Second phase | 1.7 | 100 |
| | (aqueous phase) | | |
| Nonanol | First phase | 96.8 | 69.6 |
| | Second phase | 3.2 | 30.5 |
| | (aqueous phase) | | |
| 1-Octanol | First phase | 96.9 | — |
| | Second phase | 3.1 | 100 |
| | (aqueous phase) | | |

### Results:

Phosphotungstic acids were detected in the third phases (the organic phases) of n-butyl ethyl ether and diethyl ether in amounts of 89.7% and 98.3%, respectively. Glucose was detected in the second phases (the aqueous phases) of n-butyl ethyl ether and diethyl ether in amounts of 100%.

Phosphotungstic acids were detected in the first phases (the organic phases) of 2-octanol and 1-octanol in amounts of 98.3% and 96.9%, respectively. Glucose was detected in the second phases (the aqueous phases) of 2-octanol and 1-octanol in amounts of 100%.

### Example 7

### Assay of Distribution Ratio of Phosphotungstic Acid and Glucose (Heating Experiment)

A saturated aqueous solution containing 30 g of phosphotungstic acid triacontahydrate was mixed with 5 g of glucose, the mixture was heated, and various types of the selected organic solvents were then added thereto in amounts 3 times greater than the minimal amount of the solvent necessary to dissolve phosphotungstic acid (by volume). The resultant was stirred and allowed to stand, followed by phase separation. Samples were dried using a centrifuge, the phosphotungstic acid content in the dried specimens was analyzed by the ICP mass spectrometer, and glucose content was assayed via liquid chromatography. The results are shown in Table 7 and in Table 8.

TABLE 7
Results of heating reaction test (with the use of
saturated aqueous solution of phosphotungstic acid)

| | Phase order | Distribution ratio of | Distribution |
|---|---|---|---|
| | (from top to | phosphotungstic acid | ratio of |
| | bottom) | (based on tungsten) (%) | glucose (%) |
| n-Butyl | First phase | 0.1 | — |
| ethyl ether | Second phase | 8.9 | 100 |
| | (aqueous phase) | | |
| | Third phase | 91.0 | 0 |
| Diethyl | First phase | 0.04 | 0 |
| ether | Second phase | 1.6 | 100 |
| | (aqueous phase) | | |
| | Third phase | 98.3 | 0 |

TABLE 8
Results of heating reaction test (with the use of saturated
aqueous solution of phosphotungstic acid and alcohol)

| | Phase order | Distribution ratio of | Distribution |
|---|---|---|---|
| | (from top to | phosphotungstic acid | ratio of |
| | bottom) | (based on tungsten) (%) | glucose (%) |
| 2-Ethyl-1- | First phase | 94.5 | 0 |
| hexanol | Second phase | 5.5 | 100 |
| | (aqueous phase) | | |
| 2-Octanol | First phase | 98.1 | 0.8 |
| | Second phase | 1.9 | 99.2 |
| | (aqueous phase) | | |
| Nonanol | First phase | 97.0 | 0 |
| | Second phase | 3.0 | 100 |
| | (aqueous phase) | | |
| 1-Octanol | First phase | 96.8 | 0 |
| | Second phase | 3.2 | 100 |
| | (aqueous phase) | | |

### Results:

Phosphotungstic acids were detected in the third phases (the organic phases) of n-butyl ethyl ether and diethyl ether in amounts of 91.0% and 98.3%, respectively. Glucose was detected in the second phases (the aqueous phases) of n-butyl ethyl ether and diethyl ether in amounts of 100%.

Phosphotungstic acids were detected in the first phases (the organic phases) of 2-ethyl-1-hexanol, 2-octanol, nonanol, and 1-octanol in amounts of 94.5%, 98.1%, 97.0%, and 96.8%, respectively. Glucose was detected in the second phases (the aqueous phases) of 2-ethyl-1-hexanol, 2-octanol, nonanol, and 1-octanol in amounts of 99% or more.

All publications, patents, and patent applications cited herein are incorporated herein by reference in their entirely.

1. A method for separating a heteropolyacid from a mixture containing a monosaccharide, the heteropolyacid and water using an organic solvent selected from the group consisting of linear C₂₋₄ alkyl ethyl ether and linear or branched C₆₋₁₂ alcohol.

2. The method according to claim 1, wherein the organic solvent is selected from the group consisting of n-butyl ethyl ether, diethyl ether, 2-ethyl-1-hexanol, 1-octanol, 2-octanol and nonanol.

3. The method according to claim 1, wherein the monosaccharide is glucose.

4. The method according to claim 2, wherein the monosaccharide is glucose.

5. The method according to claim 1, wherein the mixture is obtained by allowing cellulose to react with a heteropolyacid.

6. The method according to claim 2, wherein the mixture is obtained by allowing cellulose to react with a heteropolyacid.

7. The method according to claims 1, wherein the heteropolyacid is a phosphotungstic acid.

8. The method according to claims 2, wherein the heteropolyacid is a phosphotungstic acid.

9. The method according to claims 3, wherein the heteropolyacid is a phosphotungstic acid.

10. The method according to claims 4, wherein the heteropolyacid is a phosphotungstic acid.

11. The method according to claims 5, wherein the heteropolyacid is a phosphotungstic acid.

12. The method according to claims 6, wherein the heteropolyacid is a phosphotungstic acid.
